(19) United States
(12) Patent Application Publication
(10) Pub. No.: US 2019/0262180 A1
(43) Pub. Date: Aug. 29, 2019

(54) CHRONIC LOWER EXTREMITY WOUND TREATMENT MONITORING SYSTEMS AND METHODS

(71) Applicant: SuraMedical, Inc., Minneapolis, MN (US)

(72) Inventors: Jonathan Engel, Minneapolis, MN (US); Noah Rosen, Marblehead, MA (US)

(21) Appl. No.: 16/342,664

(22) PCT Filed: Oct. 19, 2017
(86) PCT No.: PCT/US2017/057380

§ 371 Date: Apr. 17, 2019

Related U.S. Application Data
(60) Provisional application No. 62/410,491, filed on Oct. 20, 2016.

(51) Int. Cl. A61F 13/00; A61F 13/06; G08B 21/20

(52) U.S. Cl. CPC A61F 13/00055; A61F 2013/00536; G08B 21/20; A61F 13/06

(57) ABSTRACT

A lower extremity wound information system can include a lower extremity wound treatment monitoring device and an external wound care information system. The monitoring device can include a moisture sensor placed at an external surface of a primary wound dressing. The moisture sensor generally would not be placed in direct contact with the wound. The monitoring device can also include a base unit placed on a posterior calf of the patient. The base unit can include a second sensor, a controller, and a communication module. The controller can receive and process information from the sensors, and determine whether to communicate information relating to the information from the sensors via the communication module to an external recipient. The external wound care information system can receive the information relating to the information from the sensors, and if appropriate, can prompt scheduling of a dressing change appointment for the patient.

### CROSS-REFERENCE TO RELATED APPLICATION

This application claims the benefit of U.S. Provisional Application No. 62/410,491 filed Oct. 20, 2016 and titled CHRONIC LEG WOUND TREATMENT MONITORING DEVICE, which is hereby incorporated by reference.

### TECHNICAL FIELD

This disclosure relates to wound treatment, and more particularly, to monitoring treatment of chronic lower extremity (leg and foot) wounds.

### BACKGROUND

Chronic (nonhealing) lower extremity wounds are a major source of patient morbidity. Two million patients present annually in the United States with a chronic lower extremity wound. The two most common reasons for the nonhealing of a lower extremity wound are chronic leg swelling (edema) and repetitive trauma to the wound, as can occur with diabetic foot wounds.

Chronic leg edema is an end-stage process seen commonly in the elderly and the obese and results from multiple etiologies including chronic post-phlebitic venous hypertension, chronic venous reflux, chronic lymphedema and poor calf muscle function. Treatment of a chronic leg wound in the setting of leg edema can include: encouraging the patient to walk; elevation of the leg when sitting; primary wound dressings that wick wound drainage away from the wound; and secondary wound dressings comprising multilayer compression bandages that compress the swollen leg.

Chronic foot wounds commonly occur in diabetics due to diabetic neuropathy that prevents the patient from sensing pain from the wound located on the plantar aspect of the foot, allowing them to repetitively traumatize the wound when walking. Treatment of a chronic diabetic foot wound can include, in addition to a primary wound dressing, an offloading device such as a total contact cast or offloading boot to limit plantar pressure and thus limit trauma to the wound with walking.

Even with proper treatment, healing a chronic lower extremity wound can be a prolonged process often taking 3 to 6 months or longer, require as many as 25 to 50 visits to a wound care provider for dressing changes (patients do not change their own dressings), and costing $10,000 to $15,000 to heal one chronic lower extremity wound. Many patients with these chronic lower extremity wounds are managed in the United States by wound care providers at the more than 1,000 wound care centers and in the home by visiting nurses.

Dilemmas of the wound care provider include determining which of the many primary/secondary dressings or offloading devices are the best for the patient and the patient's wound, and when next to schedule the patient for dressing change. A recent study looking at primary wound dressings found that 45% of primary dressing changes were performed without failure of the primary dressing (UNNECESSARY DRESSING CHANGE) and 26% of primary dressings remained in place on the patient in the home despite already have failed (PRIMARY DRESSING SATURATED) and were no longer providing therapeutic wound moisture balance (FAILED DRESSING). (Milne et al. A wearable wound moisture sensor as an indicator for wound dressing change: an observational study of wound moisture and status. Int Wound J. 2016; 13: 1309-1314.)

In addition to primary dressings, chronic leg wounds in the setting of leg edema can require adequate compression of the leg with a secondary dressing of a multilayer compression bandage. The compression bandages can fail while in place on the patient in the home and slip down the leg (especially when leg swelling decreases). Another key to reducing leg edema and speed wound healing is to encourage patients to walk and elevate when sitting. Walking stimulates the calf muscle to remove interstitial fluid. Elevation of the wounded leg when sitting also helps to remove the excess edema fluid. However, during the time between wound care appointments, objective data regarding patient compliance with walking and leg elevation are not available.

Chronic foot wounds can pose their own particular challenges. Offloading boots can be removed by the patient in the home, and when not worn subsequently during walking, healing can be impeded dramatically. Offloading total contact casts can become loose as leg swelling decreases, resulting in poor offloading and “pistoning” of the leg in the cast with walking that can lead to formation of new lower extremity wounds.

### SUMMARY

Among various aspects, the present disclosure provides a wearable monitoring device for the detection, processing, transmission and alerting of patient wound treatment compliance and wound dressing status on the patient in the home during treatment for chronic lower extremity wounds, performing multiple measurement types such as patient activity, leg elevation, primary wound dressing saturation status, compression bandage pressure changes, and offloading device pressure changes while keeping the doctor, caregivers, and patient informed wirelessly in real time.

In an illustrative but non-limiting example, the disclosure provides a lower extremity wound information system that can include a lower extremity wound treatment monitoring device and an external wound care information system. The lower extremity wound treatment monitoring device can include a moisture sensor placed at an external surface of a primary wound dressing of a wound of a patient. The moisture sensor generally would not be placed in direct contact with the wound. The lower extremity wound treatment monitoring device can also include a base unit placed on a posterior calf of the patient. The base unit can include a second sensor structured and configured to measure at least one parameter relevant to lower extremity wound healing, a controller, and a communication module. The controller can be communicatively coupled to the sensors, can be programmed and configured to receive and process information from the sensors, and programmed and configured to determine whether to communicate information relating to the information from the sensors. Such a communication can be to a recipient external to the lower extremity wound treatment monitoring device. The communication module can be functionally coupled to the controller and can be structured and configured to communicate information relating to the information from the sensors to a recipient external to the lower extremity wound treatment monitoring device. The recipient can be, for example, a patient digital device, an external wound care information system, a wound care clinic computing device, or any other suitable recipient. The external wound care information system can be programmed and configured to receive the information relating to the information from the sensors.

In another illustrative but non-limiting example, the disclosure provides a lower extremity wound information system that can include a lower extremity wound treatment monitoring device and an external wound care information system. The lower extremity wound treatment monitoring device can include at least two sensors, a controller, and a communication module. The sensors can be disposed at a patient leg and can be structured and configured to provide information about lower extremity wound healing. The controller can be communicatively coupled to the sensors, and can be programmed and configured to receive and process information from the sensors and to determine whether to communicate information relating to the information from the sensors. Such a communication can be to a recipient external to the lower extremity wound treatment monitoring device. The communication module can be functionally coupled to the controller and can be structured and configured to communicate information relating to the information from the sensors to a recipient external to the lower extremity wound treatment monitoring device. The external wound care information system can be programmed and configured to receive the information relating to the information from the sensors.

In some examples, the lower extremity wound treatment monitoring device further includes a user interface functionally coupled to the controller, a memory functionally coupled to the controller, a power supply functionally coupled to the controller, and a housing, where the user interface, the controller, the communication module, the user interface, the memory, and the power supply are each coupled to the housing. In some examples, one of the sensors is a primary dressing sensor that is external to the housing, which can be communicatively coupled to the controller via a wired connection in some cases and via a wireless connection in some cases. In some examples, one of the sensors is a mechanical load sensor. The mechanical load sensor can be integrated with the housing. In some cases, one of the sensors can be a motion sensor.

In some examples, the system can further include a personal digital device. In some such examples, the controller and the communication module can be components of the personal digital device, and the sensors can be functionally coupled to the controller via a wireless connection.

In some examples, the controller can be programmed and configured to determine that a dressing change is warranted based upon information about lower extremity wound healing from the sensors and to communicate a dressing change notification to the external wound care information system via the communication module.

In yet another illustrative but non-limiting example, the disclosure provides a method for lower extremity wound treatment. The method can include applying a dressing to a lower extremity wound of a patient and placing a lower extremity wound treatment monitoring device with the dressing. The monitoring device can include at least two sensors placed and configured to provide information about lower extremity wound healing, a controller functionally coupled to the sensors, and a communication module functionally coupled to the controller. The controller can be programmed and configured to process information about lower extremity wound healing from the sensors and to determine whether to communicate information relating to the information about lower extremity wound healing from the sensors to an external wound care information system. The communication module can be structured and configured to communicate information relating to the information about lower extremity wound healing from the sensors to the external wound care information system. The method can include the monitoring device monitoring the lower extremity wound that is dressed with the dressing. Such monitoring can include the monitoring device determining that a dressing change is warranted based upon information about lower extremity wound healing from the sensors, and the monitoring device communicating a dressing change notification to the external wound care information system.

In some examples, the method can further include the external wound care information system, responsive to receiving the dressing change notification from the monitoring device, prompting scheduling of a dressing change appointment for the patient. In some such examples, the method can further include removing the dressing, applying a subsequent dressing to the lower extremity wound, placing the lower extremity wound treatment monitoring device with the subsequent dressing, and the monitoring device monitoring the lower extremity wound that is dressed with the subsequent dressing.

In some examples of the method, the dressing can include a primary dressing that is applied in contact with the lower extremity wound, and one of the sensors can be a primary dressing sensor that is placed in contact with the primary dressing but not in contact with the lower extremity wound. The monitoring device can determine that the dressing change is warranted based upon information from the primary dressing sensor that indicates that the dressing is saturated.

In some examples of the method, the dressing can include a secondary treatment, and one of the sensors can be a secondary treatment sensor. The monitoring device's determination that the dressing change is warranted can be based upon information from the secondary treatment sensor. Further, in some such examples, the secondary treatment can be a compression dressing and the secondary treatment sensor can be placed and configured to monitor compression applied by the compression dressing. The monitoring device's determination that the dressing change is warranted can be based upon information from the secondary treatment sensor that indicates that compression applied by the compression dressing has decreased by a predetermined degree.

In some examples, the monitoring device's determination that the dressing change is warranted can be based at least in part upon a comparison of contemporary information from one of the sensors relative to initial information recorded by the one of the sensors at a time of placement of the lower extremity wound treatment monitoring device.

In some examples, one of the sensors is a motion sensor, and the method can further include recording patient activity information from the motion sensor. In some such examples, the lower extremity wound treatment monitoring device can communicate information related to the patient activity information to at least one of a patient digital device and the external wound care information system.

The above summary is not intended to describe each and every example or every implementation of the disclosure. The Description that follows more particularly exemplifies various illustrative embodiments.

### BRIEF DESCRIPTION OF THE DRAWINGS

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings, in which:

FIG. 1 is a schematic diagram of a lower extremity wound information system of the present disclosure;

FIG. 2 is a schematic block diagram of an illustrative example of a lower extremity wound treatment monitoring device;

FIG. 3a is a schematic diagram illustrating a connectivity scheme;

FIG. 3b is a schematic diagram illustrating another connectivity scheme;

FIG. 3c is a schematic diagram illustrating yet another connectivity scheme;

FIG. 4 is a schematic plan view of a top side of a base unit of a lower extremity wound treatment monitoring device;

FIG. 5 is a schematic plan view of the bottom side of the base unit of FIG. 4;

FIG. 6 is a schematic exploded perspective view of the base unit of FIG. 4;

FIG. 7 is a schematic perspective view of an illustrative example of a lower extremity wound treatment monitoring device with a base unit and an external primary dressing sensor package;

FIG. 8 is a schematic illustration showing placement of a lower extremity wound treatment monitoring device during application of a secondary compression device;

FIG. 9 is a schematic flow diagram illustrating use of a lower extremity wound treatment monitoring system of the present disclosure;

FIG. 10 is a schematic flow diagram illustrating normal operation of a lower extremity wound treatment monitoring device of the present disclosure; and

FIG. 11 is a schematic flow diagram illustrating processing of sensor data by a lower extremity wound treatment monitoring device of the present disclosure.

### DETAILED DESCRIPTION

This disclosure relates to wound treatment, and more particularly, to monitoring treatment of chronic lower extremity wounds. Various embodiments of the systems and methods are described in detail with reference to the drawings, in which like reference numerals may be used to represent like parts and assemblies throughout the several views. Reference to various embodiments does not limit the scope of the systems and methods disclosed herein. Examples of construction, dimensions, and materials may be illustrated for the various elements, but those skilled in the art will recognize that many of the examples provided have suitable alternatives that may be utilized. Any examples set forth in this specification are not intended to be limiting and merely set forth some of the many possible embodiments for the systems and methods. It is understood that various omissions and substitutions of equivalents are contemplated as circumstances may suggest or render expedient, but these are intended to cover applications or embodiments without departing from the spirit or scope of the disclosure. Also, it is to be understood that the phraseology and terminology used herein are for the purpose of description and should not be regarded as limiting.

In current practice, chronic lower extremity wounds are often treated according to the following protocol, which generally involves a series of recurring visits between a patient and a wound care provider:

In an initial visit, usually conducted at a wound care center, the patient's history is taken/considered and a physical performed. A wound care provider evaluates and debrides the wound. The wound care provider applies a primary dressing, which generally contacts the wound and which can wick or otherwise facilitate drainage and transport of exudate away from the wound. The wound care provider typically also applies a secondary treatment. In the case of a leg wound in the presence of leg edema, the secondary treatment can include a secondary dressing of a multilayer compression bandage. In the case of a foot wound, the secondary treatment can include an offloading device, which can be an offloading boot or a total contact cast.

After the initial visit, current practice then typically includes a series of follow-up visits between the patient and a wound care provider, which can take place at a wound care center, or at another location—such as a patient's home—with a visiting nurse. At a follow-up visit, the wound care provider removes the primary dressing and secondary treatment, evaluates and debrides the wound, and places a new primary dressing and secondary treatment.

A problem with current lower extremity wound treatment practice is that follow-up visits are generally scheduled according to an estimate or guess as to when a dressing change will be needed (such as when the primary dressing will be saturated or the secondary dressing will loosen), which can lead to dressing changes being performed earlier than needed (adding to overall treatment cost) or later than ideal (resulting in sub-optimal healing and added overall treatment cost).

Another component of chronic leg wound healing in the presence of leg edema is patient compliance with instructions to walk and to elevate the leg when at rest. Currently, compliance or lack thereof is self-reported by patients at the times of their follow-up visits.

In view of current chronic lower extremity wound care practice, systems and methods of the present disclosure promote and enable improved treatment of chronic lower extremity wounds, at least in part by providing objective and timely information to support decision-making and patient compliance. In some embodiments, systems and methods of the present disclosure can provide information about dressing status such that dressing changes can be scheduled based on need rather than guesswork. In some embodiments, systems and methods of the present disclosure can provide information on patient compliance, which can, for example, be used to encourage patient behavior that can improve healing.

FIG. 1 is a schematic diagram of a lower extremity wound information system 100. System 100 can include a lower extremity wound treatment monitoring device 102 that can be disposed at or on a patient's leg, generally not in contact with the lower extremity wound. Monitoring device 102 can be communicatively linked to information system elements such as a patient digital device 104, a server 106, a wound center computing device 108, and any other suitable information system device, via any appropriate communication infrastructure(s) 110. Elements of lower extremity wound information system 100 that are outside the patient's immediate vicinity, such as server 106 and wound center computing device 108, can be considered to be part of an external wound care information system. The dashed line representing communication infrastructure(s) 110 is merely schematic and should not necessarily be interpreted as limiting the communication infrastructure(s) of the present disclosure to any particular communication infrastructure topology, technology, layers, protocols, or any other aspect(s) of how communication can be implemented. The systems and methods of the present disclosure can include and be implemented by and with any communication infrastructure 110 suitable to realize the functions of the systems and methods, and should not be considered limited to any particular communication architecture unless specifically described as such.

In some embodiments of the present disclosure, lower extremity wound treatment monitoring device 102 can provide essentially current information about a lower extremity wound treatment, such as (but not limited to) whether a primary dressing is saturated and/or whether a secondary dressing is maintaining compression. Such information can be communicated via communication infrastructure 110 to any suitable recipient. Possible recipients include, in non-limiting examples, patient digital device 104, wound center computing device 108, and/or server 106, which can host a lower extremity wound treatment monitoring service. Any of patient digital device 104, wound center computing device 108, and server 106 can be considered to be part of an external wound care information system. If such information indicates that a dressing change is appropriate, then action can be taken to schedule the patient for a dressing change in the near term, such as within a day. By basing dressing changes upon actual need, increased patient throughput, cost savings, and improved healing outcomes can be achieved.

In some embodiments of the present disclosure, lower extremity wound treatment monitoring device 102 can measure, and record and/or report, patient activity, such as when and how much a patient walks, and whether the leg is elevated when at rest. In some cases, daily feedback can be provided to the patient (and in some cases, another party such as the patient's family), which can help engage the patient and allow the patient to set goals for activity for the following day. At wound care appointments, the objective measured data could be reviewed by the patient and the wound care provider. This can encourage the patient to become an active participant in the wound treatment plan.

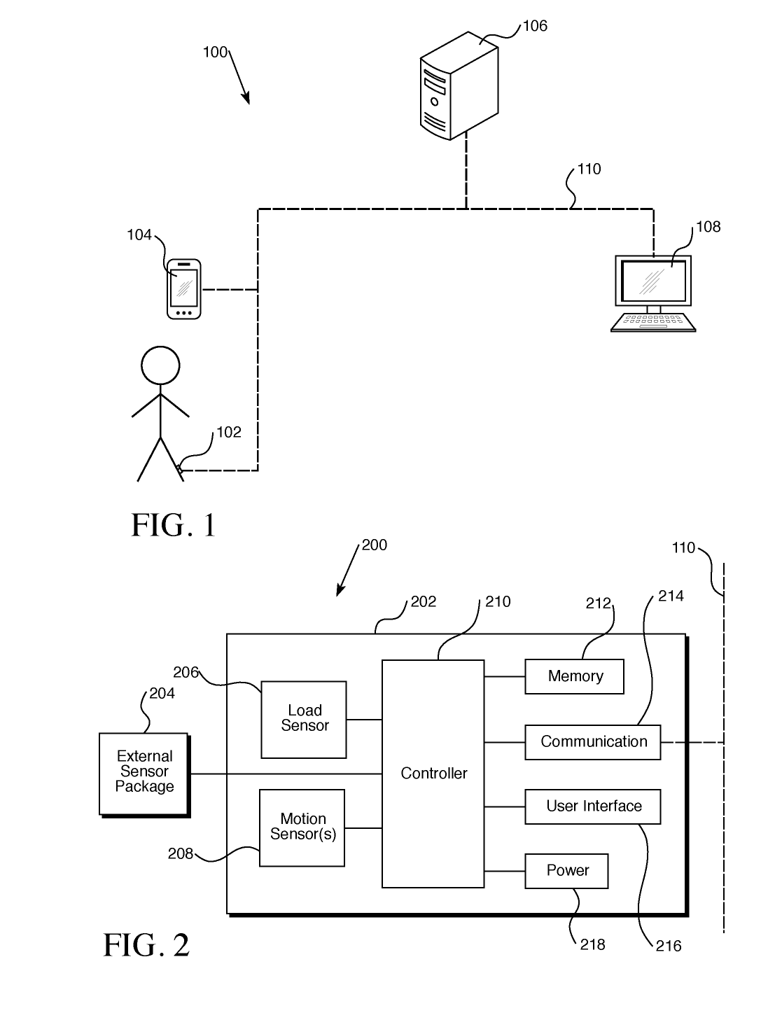

FIG. 2 is a schematic block diagram of an illustrative example of a lower extremity wound treatment monitoring device 200, which can be an example of a device like device 102 of lower extremity wound information system 100 of FIG. 1. Lower extremity wound treatment monitoring device 200 can include a housing 202, which can house components of the device, although not all components of device 200 need necessarily be housed within housing 202. For example, some components of device 200 can be external to housing 202, and some can be disposed on the housing. Housing 202 can be constructed from any suitable material(s), including plastics, polymers, metals, and so on. In some examples, housing 202 can be substantially rigid, in other examples, the housing can be substantially flexible, and in some examples, portions of the housing can be rigid and other portions can be flexible, as appropriate. The housing 202 and other components of the lower extremity wound treatment monitoring device 200 can be manufactured to be substantially water resistant such that it can robustly tolerate exposure to sweat, water, etc., and tolerate cleaning such as being wiped down between uses.

Device 200 can include one or more sensors. Device can include an external primary dressing sensor package 204, which can be configured to be applied or otherwise placed on, at, or near a primary dressing, but generally not on the wound. External primary dressing sensor package 204 can include one or more sensors for measuring characteristics relevant to a primary wound dressing, such as moisture, temperature, color, pH, chemical composition, or any other appropriate characteristics. In some embodiments, only moisture is sensed by external primary dressing sensor package 204. In some embodiments, more than one characteristic can be measured by external primary dressing sensor package 204, but only moisture information is used for dressing condition evaluation. Device 200 can include a mechanical load sensor 206 suited, for example, for measuring a parameter related to compression of a secondary dressing. In some embodiments, mechanical load sensor 206 can be integrated with housing 202 of device 200, while in some other embodiments a mechanical load sensor can be provided external to housing 202. Device 200 can also include motion sensor(s) 208, which can exploit any suitable sensing technology(ies) to detect patient motion, such as accelerometers, gyroscopes, pressure sensors, magnetic sensors, and so on. In some embodiments, device 200 can include a sensor to detect whether a lower extremity wound treatment appliance, such as an offloading boot for a foot wound, is present. Such a presence detection sensor could operate on the basis of any suitable technology. For example, the sensor could include a magnetic switch that would be closed by a magnet on an offloading boot essentially only when the boot is worn by the patient.

Device 200 can include any suitable components to receive and process information from sensors 204, 206, 208 (and any other sensors), and to communicate via information infrastructure 110, such as (but not limited to) a controller 210 programmed with any appropriate software, a memory 212, communications hardware 214, a user interface (U.I.) 216, a power source 218, and any other appropriate components. Any and/or all of these components can be coupled to housing 202 (enclosed by or otherwise attached to the housing).

Power source or sources 218 can include any suitable energy storage and/or handling devices, including (but not limited to) batteries, capacitors, fuel cells, energy harvesting devices, inductive energy transfer components, etc.

User interface 216 can include one or more buttons, switches, or other touch sensitive area(s), etc., that can allow a user to make an operational mode selection. User interface 216 also can include a display that can be configured to alert a user device status such as battery level, operation mode, communication link, etc., and may include a light emitting diode (LED) or liquid crystal display (LCD), electronic paper, or any other suitable display technology. User interface 216 can also include an audio annunciator to convey information to a user.

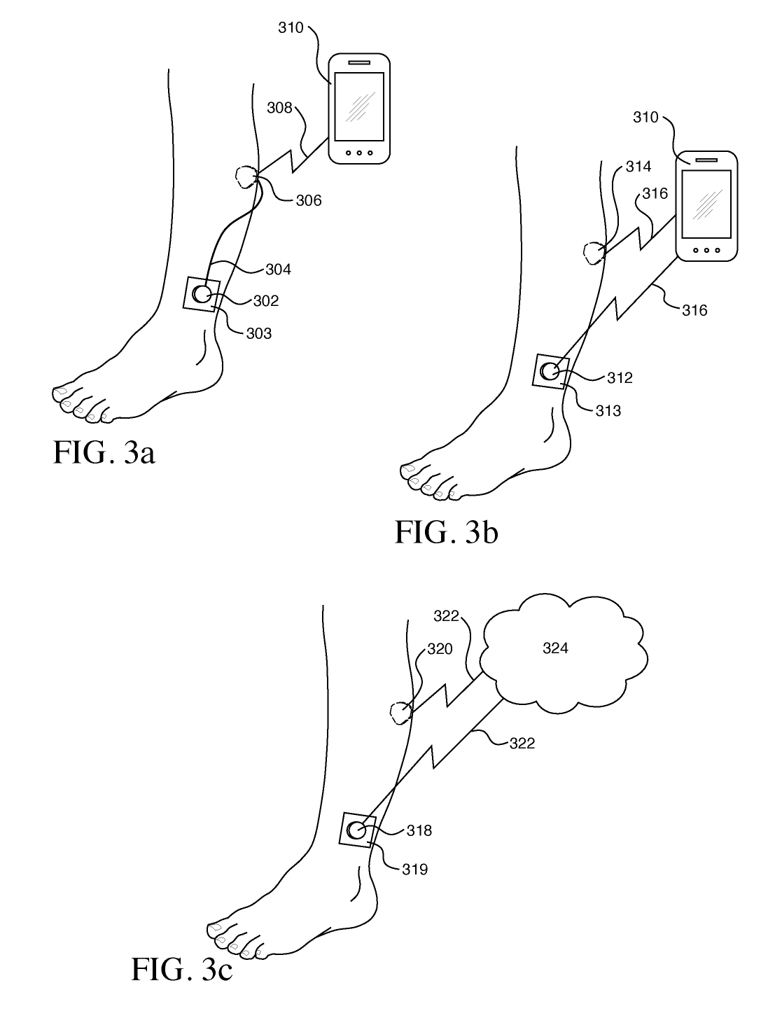

Controller 210 can be communicatively coupled to sensors 204, 206, 208 (and any other sensors) in any suitable manner, such as by wired or wireless connections. Wireless connections between sensors 204, 206, 208, etc., and controller 210 can be made via any suitable protocol, such as (but not limited to) a short-range radio frequency protocol such as a Bluetooth protocol. FIGS. 3a, 3b, and 3c schematically illustrate information connectivity schemes contemplated in the present disclosure, but these examples should not be considered limiting.

In FIG. 3a, an external sensor 302 of a lower extremity wound treatment monitoring device, which can be a primary dressing sensor package (like sensor package 204 of device 200), shown placed on a primary dressing 303 (for clarity, no further dressing are illustrated in FIGS. 3a, 3b, and 3c, but this is not limiting), can be communicatively coupled with a leg-mounted base unit 306 via a short-range link 304 which can be, for example, a wired or wireless communication link. Base unit 306 can include components of the lower extremity wound treatment monitoring device other than external sensors, and can include (like device 200), a controller, memory, communications hardware, and so on, within a housing. Base unit 306 can be coupled to additional external sensors, and can include internal or integrated sensors such as a load sensor (like sensor 206 of device 200) and/or motion sensor (like sensor 208). Components of base unit 306 can be programmed and configured to receive and process information from external sensor 302 and any other sensors of the device, and to wirelessly communicate (308) information relating to the information from the sensor(s) to a patient digital device 310, which can be like device 104 of FIG. 1. Patient digital device 310 can be a widely- and/or commercially-available multi-purpose device such as a smartphone, tablet or pad computer, personal digital assistant, or the like. Patient digital device 310 can communicate information relating to the information from the sensor(s) to other components of a lower extremity wound information system, such as an external server like server 106 of system 100 of FIG. 1, via any suitable protocol and/or technology, such as over the Internet via WiFi.

In FIG. 3b, sensor 312 (shown placed on a primary dressing 313) and sensor 314 are schematically shown being wirelessly communicatively coupled (316) directly to a patient digital device 310 without an intervening base unit 306 as in the example of FIG. 3a. In the example of FIG. 3b, sensor information processing functions can be performed by patient digital device 310 that otherwise might be performed by base unit 306 in the example of FIG. 3a. Patient digital device 310 can communicate information relating to the information from sensors 312, 314 to other components of a lower extremity wound information system, such as an external server. In embodiments where there is not a base unit such as unit 306, and in which a patient digital device provides the controller and communications hardware for a lower extremity wound treatment monitoring device, the patient digital device may be considered not to be part of an external wound care information system.

In FIG. 3c, sensor 318 (shown placed on a primary dressing 319) and sensor 320 are schematically shown being wirelessly communicatively coupled (322) to components of a lower extremity wound information system that is remote from a patient location, represented in cartoon form by “cloud” 324. In this example, sensors 318 and 320 can be coupled to remote information resources such as server 106 of FIG. 1 without an intervening base unit (such as unit 306 of FIG. 3a) or personal digital device (such as devices 310 of FIGS. 3a and 3b). Sensors 318 and 320 can communicate by any suitable technology and/or protocol, for example, via a WiFi router, a mesh network, or any other suitable communication infrastructure. In the example of FIG. 3c, sensor information processing functions can be performed remotely from the patient, such as at a server such as server 106 of FIG. 1.

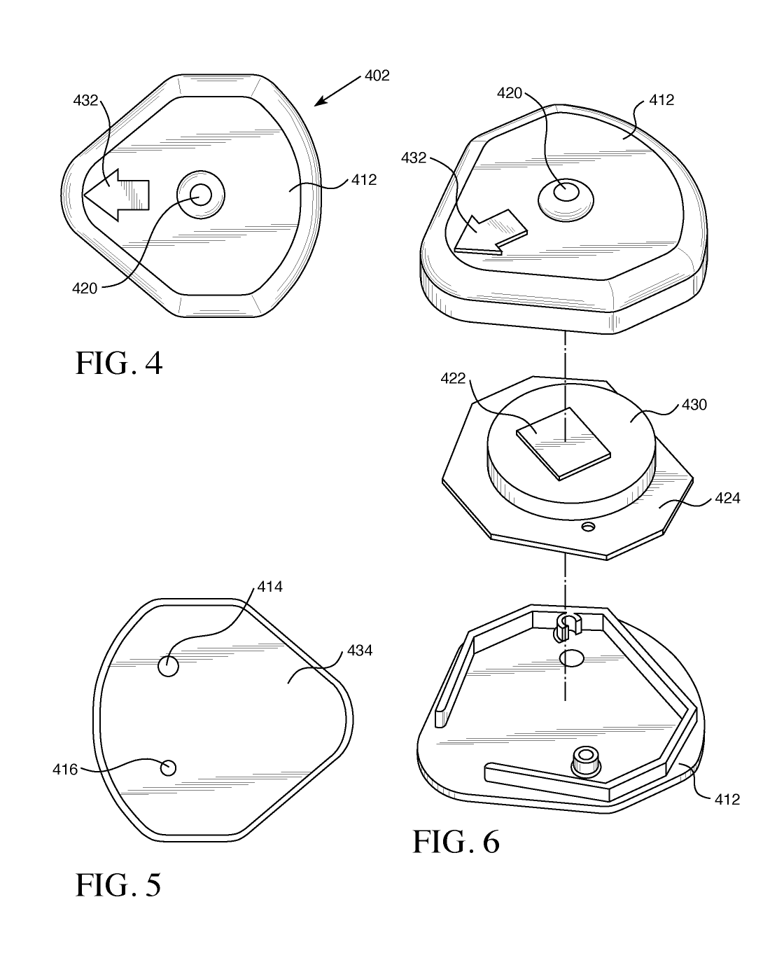

FIGS. 4, 5, and 6 are schematic illustrations of an illustrative example of a base unit 402 (similar to or like base unit 306 of FIG. 3a and with components similarly or identically as described for device 200 of FIG. 2) of an embodiment of a lower extremity wound treatment monitoring device, which can be like device 200 of FIG. 2. FIG. 4 is a schematic plan view of a top side of base unit 402, where “top” can refer to a side of the base unit that faces away from a patient's leg when the base unit is secured relative to the leg. FIG. 5 is a schematic plan view of a bottom side of base unit 402, and FIG. 6 is a schematic exploded perspective view of base unit 402.

Base unit 402 can include a housing 412 and a user interface that includes an input device 414 such as a pushbutton or other touch sensitive area that can be configured to receive an operational mode selection and any other appropriate user input. The user interface can also include a user display 416 such as an LED, or any other suitable display element, that can alert the user to device status such as battery level, operation mode, etc. As described in reference to device 200, base unit 402 can include an audio output (not shown in FIG. 4-6) configured to indicate operational information. Housing 412 can include a load transferring element 420 that can facilitate transfer of external force, for example from an overlaying compression wrap treatment device, onto a load sensor 422 that can be located within housing 412, for example on an internal component board 424. In some other embodiments, a load sensor can be located on the housing, at any suitable location (for example, at the location of load transferring element 420 of base unit 402). Component board 424 can include and can structurally support any appropriate components of base unit 402, such as a power supply, a controller, a memory, communication hardware, sensors, etc. The described arrangement of components on board 424 should not be considered limiting, however, and other arrangements are possible, including mounting of components on multiple component boards, mounting of components on interior or exterior surfaces of housing 412, and any other suitable and functional arrangement.

The exterior of housing 412 can include an orientation indicator 432 to assist a caregiver in proper placement of base unit 402, although in some embodiments, motion/orientation sensors of the base unit (such as sensors 208) can be used to detect and calculate device orientation regardless of placement on the patient. Base unit 402 can include one or more non-skid feature(s) 434 to prevent or reduce migration of the unit during wear. Non-skid feature(s) 434 can include raised bumps, a sticky surface, or other methods or materials that will hold the base unit 402 substantially in place without undue discomfort to the patient.

For comfortable wear by the patient, the base unit (702, 402, 306) can be made to be as small as practical. In some embodiments, the thickness of the base unit can be less than about 10 mm. The lateral dimensions of the base unit can be about 25 mm to 50 mm, or less. In some embodiments, housing 412 can be substantially rigid, and in other embodiments, the housing and potentially other components of a base unit (such as, but not limited to, a circuit board, electronic components, and a battery) can be substantially flexible, which may allow a caregiver greater freedom in placing the base unit with a patient without causing patient discomfort. In some embodiments, components of a lower extremity wound treatment monitoring device can be integrated into dressing fabric. In some embodiments, a complete monitoring device can be provided integrated with a wound dressing. In some other embodiments, a moisture sensor can be provided integrated with a wound dressing.

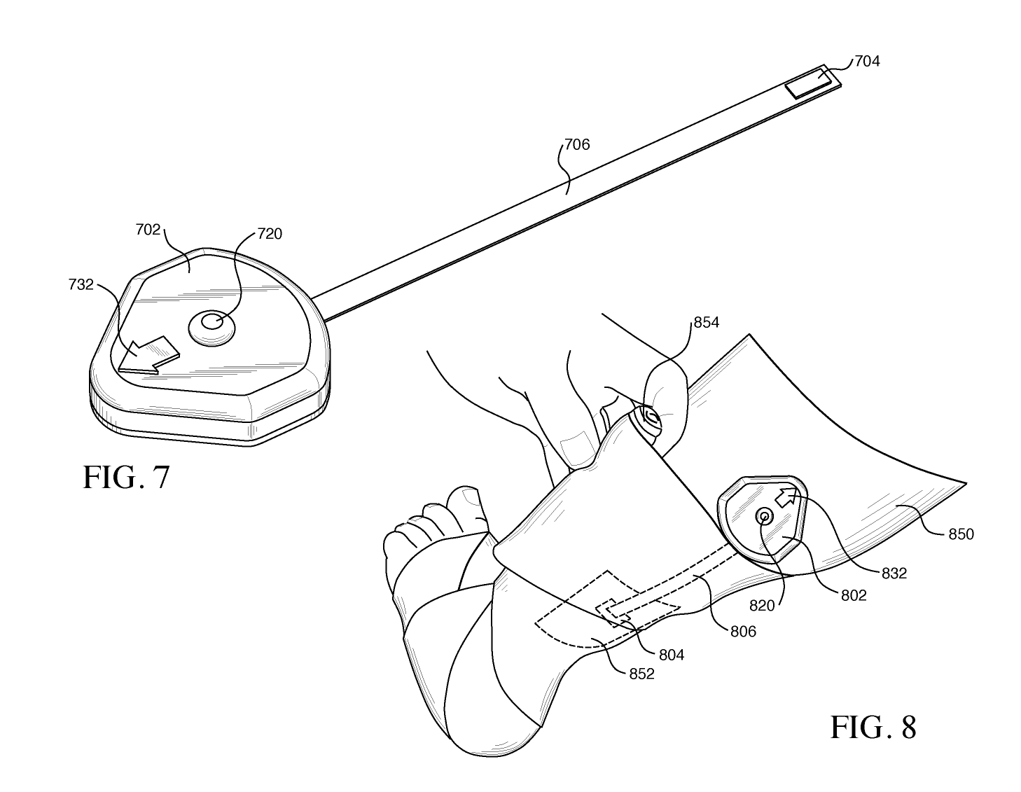

FIG. 7 is a schematic perspective view of an illustrative example of a monitoring device 700 that can include a base unit 702 (similar to or like base unit 402), and an external primary dressing sensor package 704 (similar to or like package 204 of device 200), which is illustrated as being communicatively coupled to the base unit via a wired connection 706. In some other embodiments, base unit 702 can be communicatively coupled to an external primary dressing sensor package wirelessly (not shown in FIG. 7). External primary dressing sensor package 704 and/or other external primary dressing sensor package(s) can enable assessment of the primary wound dressing by providing moisture sensing (for detecting dressing saturation).

FIG. 8 is a schematic illustration showing placement of a lower extremity wound treatment monitoring device during application of a secondary compression device 850. Before the moment in time illustrated in FIG. 8, a primary wound dressing 852 has already been placed and is not directly visible (location indicated in dashed lines). External primary dressing sensor package 804 (not directly visible, but indicated in dashed lines) can be placed at or over primary wound dressing 852 (e.g., on an external surface of the dressing opposite the wound). In general, primary dressing sensor packages of the present disclosure are not placed in direct contact with a wound, in the interest of minimizing trauma to the wound. External primary dressing sensor package 804 can be communicatively coupled to base unit 802 of the lower extremity wound treatment monitoring device via wired connection 806 (not directly visible, but indicated in dashed lines), but in some embodiments, it can be communicatively coupled wirelessly. In some embodiments, a lower extremity wound treatment monitoring device may not necessarily include an external primary dressing sensor.

In some examples and as illustrated in FIG. 8, base unit 802 (and other base units of lower extremity wound treatment monitoring devices of the present disclosure) can be placed on the back or posterior of the calf muscle of the leg. Generally, base units of lower extremity wound treatment monitoring devices of the present disclosure are not placed on or over a wound, in order to prevent additional damage to the wound area. The posterior of the calf can be a favorable position for the base unit compared to alternative locations such as over bone (e.g, the shin), where the base unit could cause discomfort. Orientation indicator 832 can indicate proper orientation for base unit 802, although in some embodiments, position/orientation sensors of the base unit in combination with suitable programming of the controller of the unit can calculate and/or compensate for orientation, relaxing or eliminating requirements for particular orientation at time of placement.

Application of a secondary treatment can proceed before, after, and/or concurrently with placement of base unit 802. In the illustrative example of FIG. 8, the secondary treatment can include a compression bandage 850, which is illustrated as being manually unrolled from a spool 854.) At least one layer of compression bandage can be applied beneath base unit 802 (i.e., between the base unit and the patient's skin) to improve patient comfort. One or more overlaying layers of compression bandage 850 can help hold the base unit 802 in place with the help of non-skid feature(s) of the base unit. One or more overlaying layers of compression bandage 850 can exert force on load transferring element 820, which can transmit force to a load sensor housed within base unit 802.

As discussed elsewhere herein, other secondary treatments are possible, such as a total contact cast or offloading boot. In such cases, a base unit of a lower extremity wound treatment monitoring device can be held in place at least in part by the total contact cast or a stockinette (if an offloading boot is to be used), and/or another component, to maintain the device in an appropriate location where it does not traumatize the wound or cause other irritation to the patient.

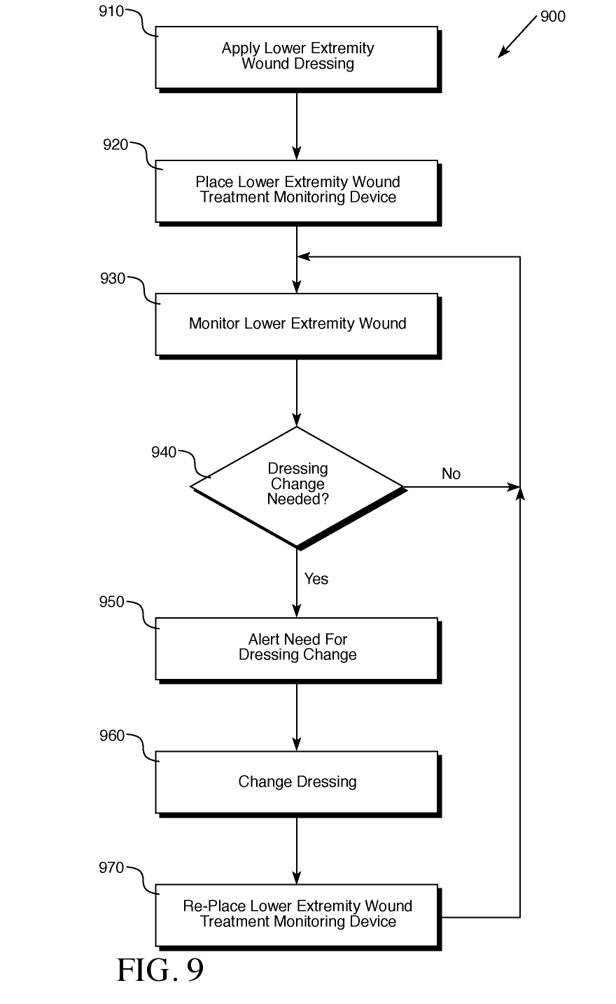

FIG. 9 is a schematic flow diagram illustrating use of a lower extremity wound treatment monitoring system of the present disclosure. At an initial visit, a lower extremity wound dressing can be applied at 910. Application 910 of the lower extremity wound dressing can be performed following evaluation of a patient's history and debridement of the wound. At 920, the lower extremity wound treatment monitoring device can be placed. Placement 920 of the lower extremity wound treatment monitoring device may be performed in conjunction with dressing the wound. For example, after a primary wound dressing has been applied, a primary dressing sensor package (204, 302, 704) can be placed on, with, or over the primary wound dressing. A base unit (306, 402, 702) of a lower extremity wound treatment monitoring device can be placed in relation to a secondary treatment as described elsewhere herein.

Initial placement 920 of the lower extremity wound treatment monitoring device can also include any other steps or actions needed or desirable for initialization of the device. Such actions can include powering up of the device (e.g., via user interface like U.I. 216), and linking the device to either or both of application software (“app”) executing on a clinical computing device (e.g., device 108) and/or a patient digital device (e.g., device 104). In some examples, a clinical app can be populated with patient information, either manually or automatically (for example, in coordination with an electronic medical records or patient information system). The clinical app also can be configured with any suitable device operation parameters. The clinical app can download any appropriate information and/or parameters to the lower extremity wound treatment monitoring device as part of initialization, as well as any firmware updates, etc.

Another action than can be taken as part of placement 920 to initialize the device can be recording initial or reference values of sensor readings. With the patient's leg down (e.g., in a standing position) and the base unit properly oriented (e.g., with orientation indicator 432, 732, 832 pointing up, and/or internal orientation sensors of the device indicating appropriate orientation), an initial set of sensor readings can be recorded (e.g., via command of the clinical app or via the user interface of the wound treatment monitoring device). The recorded initial sensor readings of quantities such as wound dressing moisture, secondary dressing compression, device inertia, etc., can provide reference values against which subsequent readings can be compared, as discussed further elsewhere herein.

Method 900 can include monitoring 930 of the patient's lower extremity wound with the lower extremity wound monitoring device, which can be part of a lower extremity wound monitoring system (such as or like system 100 of FIG. 1). Aspects of monitoring a lower extremity wound are discussed in further detail elsewhere herein, and can include, for example, monitoring a moisture level of a primary wound dressing to determine whether the dressing is saturated, which generally could or would indicate a need for a dressing change. At 940, method 900 can include determining whether a dressing change is needed. Such a determination 940 could be made by any suitable controller or processor of a lower extremity wound monitoring system such as, but not limited to, a controller of a base unit (702, 402, 306), a patient digital device 104, a server 106, or wound center computing device 108.

If it is determined at 940 that a dressing change is not warranted, monitoring can continue at 930. If a dressing is warranted, then at 950 an alert can be issued for a need for a dressing change. In some embodiments, such an alert can be issued by a base unit (702, 402, 306) of a lower extremity wound monitoring device, and received by an external wound care information system (for example, at server 106), with the receiving system then prompting scheduling of a dressing change appointment for the patient (for example, by notifying a wound care center and/or the patient that a dressing change appointment is called-for).

At a resultant dressing change appointment prompted by the monitoring system, method 900 can include a dressing change 960. Also at the dressing change appointment, the lower extremity wound treatment monitoring device can be removed, cleaned if necessary, and re-placed with the changed dressing at 970. The re-placement of the lower extremity wound treatment monitoring device can include taking a new set of initial sensor readings for later comparison. Download of data from the lower extremity wound treatment device, for example via a clinical computing device, can also be performed at a dressing change appointment.

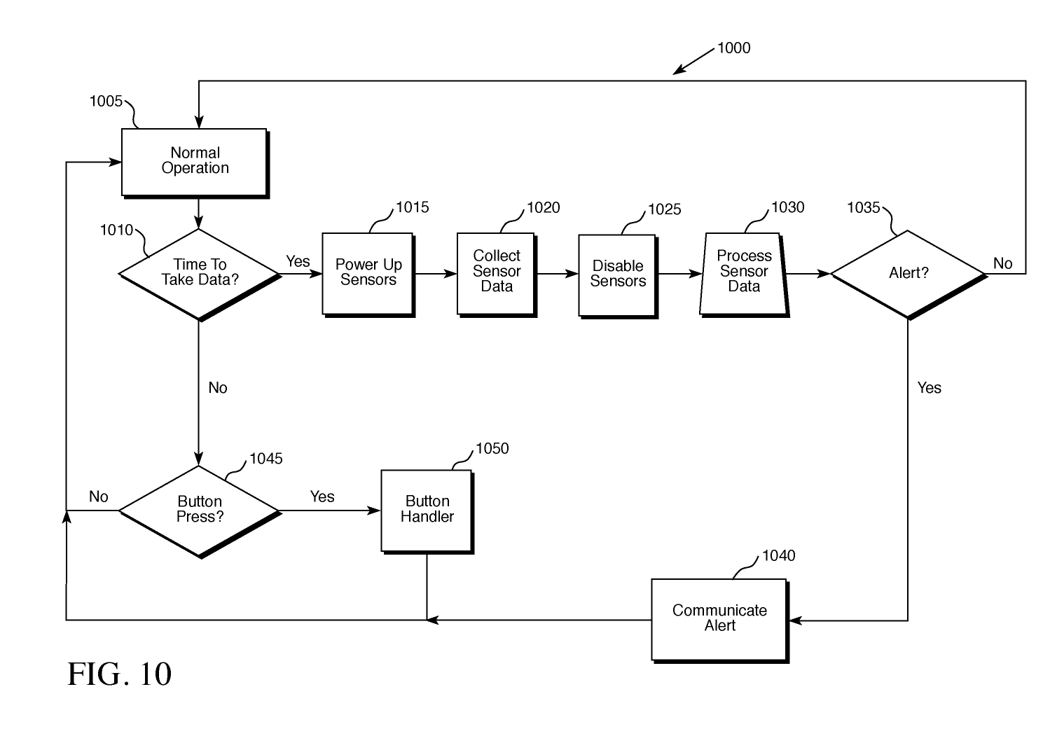

FIG. 10 is a schematic flow diagram illustrating normal operation 1000 of a lower extremity wound treatment monitoring device of the present disclosure, as for example may occur during monitoring 930 of method 900. After the beginning 1005 of a cycle of normal operation 1000, the controller of a lower extremity wound monitoring device can determine whether it is time to take data with the devices sensors. Data can be taken at regular, predetermined time intervals, or at any other appropriate time intervals. Frequency of data-taking can be balanced with the need to manage the device power resources, which may be finite. In some cases, data-taking frequency can be increased dynamically, such as less frequently when sensor measurements have not recently changed significantly, and more frequently when recent sensor measurements indicate changes, for example, in wound dressing condition(s).

If it is not time to take data, normal operation can proceed to user-interface handling, including determining whether a U.I. button has been pressed at 1045, and if so, handling the button press at 1050. Normal operation can then return to the beginning of a cycle at 1005.

If it is time to take data, sensors can be powered up at 1015, data collected from the sensors at 1020, and sensors disabled (e.g., powered-down) at 1025. At 1030, sensor data can be processed, as described further elsewhere herein. At 1035, the controller of the lower extremity wound monitoring device can determine whether an alert should be communicated to the external wound care information system, for example to a patient digital device 104, a wound center computing device 108, and/or a server 106. If so, the alert can be communicated at 1040, then normal operation can then return to the beginning of a cycle at 1005. Possible alerts can include, for example and without limitation, an alert that a dressing change is warranted, or an alert to a patient that they should, if inactive, increase the time walking or elevate the leg when sitting.

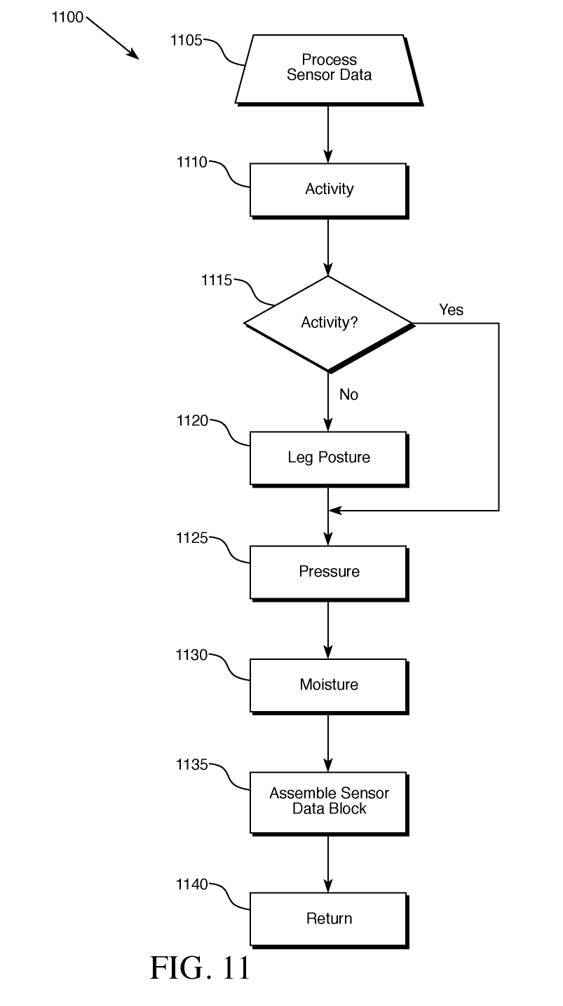

FIG. 11 is a schematic flow diagram illustrating processing of sensor data 1100 by a lower extremity wound treatment monitoring device of the present disclosure, as for example may occur at 1030 of normal operation 1000 of the device. Processing sensor data can include an algorithm at 1110 to determine whether the patient is engaged in physical activity that involves leg motion, as detected, for example, by motion sensors of the device. If activity is not sensed, the method can include an algorithm for checking leg posture at 1120, which can include comparing current leg orientation measurements from motion sensors with measurements taken at the time of device initialization (e.g., when the device is placed at the time of wound dressing). If the orientation measurement indicates that the leg is down during a period of no activity, the controller of the device could subsequently determine, based on such data, to alert the patient that it would be desirable to elevate the leg.

Processing of sensor data 1100 can also include algorithms for evaluating data from sensors relating to pressure or force (at 1125) applied by a secondary treatment, such as a compression bandage, and primary dressing moisture (at 1130), both of which can include comparing contemporary measurements from the relevant sensors with measurements taken at the time of device initialization. In lower extremity wound monitoring devices of the present disclosure, the use of such relative sensor measurement comparisons can obviate the need to provide absolute calibrated measurements for assessing dressing status. If contemporary measurements of pressure and/or moisture differ from initial measurements by greater than a predetermined threshold, then the changed characteristic can be flagged as (potentially) warranting an alert for communication external to the device.

At 1135, processing of sensor data 1100 can include assembling a sensor data block, which can be recorded into device memory and can be evaluated in element 1035 of normal operation 1000 with regard to whether an external alert should be communicated. The data block can include raw and/or processed sensor information, timestamp(s), device parameters such as battery status, and any other appropriate information. While it is anticipated that a leg treatment device power supply can be provided that can supply sufficient energy to support function of the device for the entire course of a patient's treatment, a power low alert function can be provided for contingencies.

In some embodiments, lower extremity wound treatment monitoring devices can be used in a standalone mode, in which a patient's lower extremity wound treatment monitoring device is not communicatively linked to external elements of a wound care information system such as a patient digital device 104, a wound center computing device 108, or a server 106. In some examples of standalone implementations, dressing change notifications or alerts can be made via the user interface 216, for example by a visual indication (e.g., an LED) or audible annunciation, prompting a patient to schedule a dressing change. In such standalone implementations, data can be logged in a memory of the lower extremity wound treatment device and subsequently downloaded upon a patient's next visit (whether at a wound care center, or home) for a dressing change. The downloaded information can be reviewed by the caregiver and patient at that time.

Lower extremity wound treatment apps on a patient's digital device 104 and/or a wound center computing device 108 can process incoming information from the lower extremity wound treatment monitoring device, present it for inspection, display configurable alerts, and allow the caregiver, physician, patient, or other appropriate user to view, store, manipulate and otherwise interact with the health information. An app on the patient's digital device 104 can help promote leg healing between wound dressing change appointments based upon collected data. For example, such an app could remind a patient to elevate their leg when not active, to put on an offloading boot, if used, when initiating activity, and to promote activity generally, with such encouragement informed by measured data about a patient's actual activity history.

Functions provided by lower extremity wound information systems of the present disclosure can include charting of health information over time, alerting the user or physician of treatment compliance goals (steps taken, elevation duration, etc.) as well as alerting the physician of treatment status such as compression device pressure or wound dressing status (saturation, pH, temperature, etc.).

In broad overview, the present disclosure provides a medical monitoring system capable of detecting treatment status and patient compliance data during the treatment of chronic lower extremity wounds, including leg wounds in the presence of leg edema and chronic foot wounds. The system can provide health information (based upon actual measurements) useful to healthcare providers for the tracking and active adjustment of treatment as well as tracking patient compliance with treatment goals. This can be accomplished by the analysis of data captured from internal sensors and/or external sensor for detecting various patient status, compression device, and wound dressing parameters. Algorithms can turn such raw data into health information that the healthcare provider can evaluate to improve treatment. This health information can be stored on the monitoring device for access when in the clinic or the health information can be transmitted wirelessly to the healthcare provider in near real time for constant monitoring.

Persons of ordinary skill in arts relevant to this disclosure and subject matter hereof will recognize that embodiments may comprise fewer features than illustrated in any individual embodiment described by example or otherwise contemplated herein. Embodiments described herein are not meant to be an exhaustive presentation of ways in which various features may be combined and/or arranged. Accordingly, the embodiments are not mutually exclusive combinations of features; rather, embodiments can comprise a combination of different individual features selected from different individual embodiments, as understood by persons of ordinary skill in the relevant arts. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of Section 112, sixth paragraph of 35 U.S.C. are not to be invoked unless the specific terms “means for” or “step for” are recited in a claim.

What is claimed is:

1. A lower extremity wound information system, comprising:
a lower extremity wound treatment monitoring device, including:
a moisture sensor placed at an external surface of a primary wound dressing of a wound of a patient, the moisture sensor placed not in direct contact with the wound;
a base unit placed on a posterior calf of the patient, the base unit including:
a second sensor structured and configured to measure at least one parameter relevant to lower extremity wound healing;
a controller communicatively coupled to the moisture sensor and the second sensor, the controller programmed and configured to receive and process information from the moisture sensor and the second sensor and to determine whether to communicate information relating to the information from the moisture sensor and the second sensor, such communication being to a recipient external to the lower extremity wound treatment monitoring device; and
a communication module functionally coupled to the controller, the communication module structured and configured to communicate information relating to the information from the moisture sensor and the second sensor to the recipient external to the lower extremity wound treatment monitoring device; and
an external wound care information system programmed and configured to receive the information relating to the information from the moisture sensor and the second sensor.

2. A lower extremity wound information system, comprising:
a lower extremity wound treatment monitoring device, including:
at least two sensors disposed at a patient leg, the at least two sensors structured and configured to provide information about lower extremity wound healing;
a controller communicatively coupled to the at least two sensors, the controller programmed and configured to receive and process information from the at least two sensors and to determine whether to communicate information relating to the information from the at least two sensors, such communication being to a recipient external to the lower extremity wound treatment monitoring device; and
a communication module functionally coupled to the controller, the communication module structured and configured to communicate information relating to the information from the at least two sensors to the recipient external to the lower extremity wound treatment monitoring device; and
an external wound care information system programmed and configured to receive the information relating to the information from the at least two sensors.

3. The system of claim 2, wherein the lower extremity wound treatment monitoring device further includes:
a user interface functionally coupled to the controller;
a memory functionally coupled to the controller;
a power supply functionally coupled to the controller; and
a housing,
wherein the user interface, the controller, the communication module, the user interface, the memory, and the power supply are each coupled to the housing.

4. The system of claim 3, wherein one of the at least two sensors is a primary dressing sensor that is external to the housing.

5. The system of claim 4, wherein the primary dressing sensor is communicatively coupled to the controller via a wired connection.

6. The system of claim 4, wherein the primary dressing sensor is communicatively coupled to the controller via a wireless connection.

7. The system of claim 3, wherein one of the at least two sensors is a mechanical load sensor.

8. The system of claim 7, wherein the mechanical load sensor is integrated with the housing.

9. The system of claim 3, wherein one of the at least two sensors is a motion sensor.

10. The system of claim 2, wherein the system further includes a personal digital device, and further wherein:
the controller and the communication module are components of the personal digital device; and
the at least two sensors are functionally coupled to the controller via a wireless connection.

11. The system of claim 2, wherein the controller is programmed and configured to determine that a dressing change is warranted based upon information about lower extremity wound healing from the at least two sensors, and to communicate a dressing change notification to the external wound care information system via the communication module.

12. A method for lower extremity wound treatment, comprising:
applying a dressing to a lower extremity wound of a patient;
placing a lower extremity wound treatment monitoring device with the dressing, wherein the monitoring device includes:
at least two sensors placed and configured to provide information about lower extremity wound healing;
a controller functionally coupled to the at least two sensors, the controller programmed and configured to process information about lower extremity wound healing from the at least two sensors and to determine whether to communicate information relating to the information about lower extremity wound healing from the at least two sensors to an external wound care information system; and
a communication module functionally coupled to the controller, the communication module structured and configured to communicate information relating to the information about lower extremity wound healing from the at least two sensors to the external wound care information system; and
the monitoring device monitoring the lower extremity wound that is dressed with the dressing, the monitoring including:
the monitoring device determining that a dressing change is warranted based upon information about lower extremity wound healing from the at least two sensors; and
the monitoring device communicating a dressing change notification to the external wound care information system.

13. The method of claim 12, further comprising the external wound care information system, responsive to receiving the dressing change notification from the monitoring device, prompting scheduling of a dressing change appointment for the patient.

14. The method of claim 13, further comprising:
removing the dressing;
applying a subsequent dressing to the lower extremity wound;
placing the lower extremity wound treatment monitoring device with the subsequent dressing; and
the monitoring device monitoring the lower extremity wound that is dressed with the subsequent dressing.

15. The method of claim 12, wherein:
the dressing includes a primary dressing that is applied in contact with the lower extremity wound;
one of the at least two sensors is a primary dressing sensor that is placed in contact with the primary dressing but not in contact with the lower extremity wound; and
the monitoring device determining that the dressing change is warranted is based upon information from the primary dressing sensor that indicates that the dressing is saturated.

16. The method of claim 12, wherein:
the dressing includes a secondary treatment;
one of the at least two sensors is a secondary treatment sensor; and
the monitoring device determining that the dressing change is warranted is based upon information from the secondary treatment sensor.

17. The method of claim 16, wherein:
the secondary treatment is a compression dressing;
the secondary treatment sensor is placed and configured to monitor compression applied by the compression dressing; and
the monitoring device determining that the dressing change is warranted is based upon information from the secondary treatment sensor that indicates that compression applied by the compression dressing has decreased by a predetermined degree.

18. The method of claim 12, wherein the monitoring device determining that the dressing change is warranted is based at least in part upon a comparison of contemporary information from one of the at least two sensors relative to initial information recorded by the one of the at least two sensors at a time of placement of the lower extremity wound treatment monitoring device.

19. The method of claim 12, wherein one of the at least two sensors is a motion sensor, the method further comprising recording patient activity information from the motion sensor.

20. The method of claim 19, further comprising the lower extremity wound treatment monitoring device communicating information related to the patient activity information to at least one of a patient digital device and the external wound care information system.
